(19) United States
(12) Patent Application Publication
(10) Pub. No.: US 2009/0008268 A1
(43) Pub. Date: Jan. 8, 2009

(54) Process for Production of a Disinfectant Through the Electrochemical Activation (Eca) of Water, a Disinfectant Produced in this Way and the Use Thereof

(76) Inventors: Peter Salathe, Liestal (CH); Christian Fischer, Wald-Michelbach (DE); Bernd Jost, Abtsteinach (DE); Steven Gross, Beerfelden (DE); Volkmar Schmidt, Viernheim (DE)

Correspondence Address: PATENTANWAELTE LICHTI + PARTNER GBR, POSTFACH 41 07 60, D-76207, KARLSRUHE, DE

(21) Appl. No.: 12/223,818

(22) PCT Filed: Feb. 14, 2007
(86) PCT No.: PCT/EP2007/001265

§ 371 Date: Aug. 11, 2008

(30) Foreign Application Priority Data
Feb. 17, 2006 (DE) 10 2006 007 931.0
Sep. 11, 2006 (DE) 10 2006 043 267.3

(51) Int. Cl. C02F 1/461

(52) U.S. Cl. 205/746

(57) ABSTRACT

A description is given of a process for the production of a disinfectant by electrochemical activation (ECA) of water, in that to the water to be disinfected is added an electrolytic solution, particularly a sodium or potassium chloride solution and the water to which the electrolytic solution has been added in the form of a dilute water/electrolytic solution is supplied with an electrical current in an electrolytic reactor with a cathode compartment having a cathode and with an anode compartment having an anode separated spatially from the cathode compartment by applying a d.c. voltage to the electrodes, in order to bring the water/electrolytic solution into a metastable state suitable for disinfection. To bring about a reliable disinfecting action of the electrochemically activated water and with high reproducibility, the invention proposes that the pH-value of the dilute water/electrolytic solution in the reactor anode compartment is controlled to a value between 2.5 and 3.5, particularly approximately 3, so that the potential of the anodic oxidation is controlled (potential-controlled anodic oxidation or PAO). The invention also relates to a disinfectant produced in this way and the use thereof.

The invention relates to a process for the production of a disinfectant through the electrochemical activation (ECA) of water, in that to the water is added an electrolytic solution, particularly a sodium and/or potassium chloride solution and the water supplied with the electrolytic solution in the form of a dilute water/electrolytic solution is subject to the action of an electrical current in an electrolytic reactor having at least one cathode compartment with a cathode and having at least one anode compartment with an anode separated spatially from the cathode compartment, particularly by means of a diaphragm or membrane by applying a d.c. voltage to the electrodes in order to bring the water/electrolytic solution into a metastable state suitable for disinfection. The invention also relates to a disinfectant produced in this way and the use thereof.

The electrochemical activation or treatment process is particularly known in connection with the disinfection of water. A dilute solution of an electrolyte, particularly a neutral salt, such as sodium chloride (NaCl) or common salt, potassium chloride (KCl) or the like is brought into an active state suitable for disinfection in an electrolytic reactor by applying a voltage to its electrodes and which is generally of a metastable nature and as a function of the water and the process parameters used can last for a long time. The electrolytic reactor has a cathode compartment with one or more cathodes and an anode compartment with one or more anodes, the anode compartment and cathode compartment being separated spatially from one another by means of an electrically conductive, particularly an ion-conductive, diaphragm or by means of a membrane with the indicated characteristics, in order to prevent a mixing of the water/electrolytic solution present in both compartments. Whilst during electrolysis generally a substantially complete conversion of the educts used—in the case of using a sodium chloride solution to chlorine gas (Cl₂) and caustic soda solution (NaOH), in the case of using a potassium chloride solution to chlorine gas and caustic potash solution (KOH)— is sought using highly concentrated electrolytic solutions in order to maximize the chlorine gas yield, in the case of electrochemical activation the water/electrolytic solution is supplied to the electrolytic reactor in a much more dilute form, generally in a concentration of max 20 g/l, preferably max 10 g/l and is only converted to a very limited extent in order to advantageously modify the physical and chemical characteristics of the solution and in particular increase the redox potential of the water mixed with the electrolyte, so that a disinfecting action is obtained. Correspondingly, in the case of electrochemical activation, the reaction conditions, such as pressure, temperature, electrode current, etc. are generally chosen in a more moderate form than for chlorine-alkali electrolysis. It is advantageous with such an electrochemical treatment, which is referred to in the scope of the present application as “electrochemical activation”, that the substances used in their given concentrations and which are also authorized according to the German Drinking Water Ordnance, have a particularly good health and environmental compatibility.

As with electrolysis, also with electrochemical activation oxidation takes place at the anode, (i.e. at the positively charged electrode), whereas a reduction takes place at the cathode (i.e. at the negatively charged electrode). When using a dilute neutral salt solution, such as a sodium chloride solution, mainly hydrogen is produced at the cathode in accordance with the following reaction equation (1):

2H₂O+2e⁻--->H₂+2OH⁻ (1)

which is e.g. removed from the reactor cathode compartment after gassing out from the solution. In addition, the dilute water/electrolytic solution becomes alkaline in the electrolytic reactor cathode compartment through the formation of hydroxide ions.

According to the following reaction equations (2) and (3), at the anode is more particularly produced the chemical oxidants oxygen (O₂) and chlorine (Cl₂), which are known to be effective regarding a disinfection of water. It must also be borne in mind that as a result of the formation of H₃O⁺ ions the dilute water/electrolytic solution in the electrolytic reactor anode compartment becomes acid:

6H₂O--->O₂+4H₃O⁺+4e⁻ (2),

2Cl⁻--->Cl₂+2e⁻ (3).

Chlorine dissociates in water in accordance with the following equilibrium reaction (4) in hypochlorite ions (OCl⁻) and chloride ions (Cl⁻), which can react with a suitable cation, e.g. Na⁺ from the electrolyte, or with a proton or a H₃O⁺ ion to the corresponding (sodium) salt or to the corresponding acid, i.e. to hypochlorous acid (HOCl) and hydrogen chloride or dilute hydrochloric acid (HCl):

Cl₂+3H₂O<===>2H₃O⁺+OCl⁻+Cl⁻ (4).

By secondary reactions further substances are produced from the aforementioned substances formed at the anode and they are also known to be active regarding the disinfection of water. These are in particular hydrogen peroxide (H₂O₂), reaction equation (5)), ozone (O₃, reaction equation (6)), chlorine dioxide (ClO₂, reaction equation (7)), chlorates (ClO₃⁻, reaction equation (8)) and various radicals (reaction equations (9) an (10)).

4H₂O--->H₂O₂+2H₃O⁺+2e⁻ (5)

0₂+3H₂O--->O₃+2H₃O⁺+2e⁻ (6)

Cl⁻+4OH⁻--->ClO₂+2H₂O+5e⁻ (7)

3OCl⁻---->ClO₃⁻+2Cl⁻ (8),

5H₂O--->HO₂.+3H₃O⁺+3e⁻ (9),

H₂O₂+H₂O--->HO₂.+H₃O⁺+e⁻ (10).

A disadvantage of the electrochemical activation process is the lack of quality control, because the usually empirically determined process parameters necessary for an adequate water disinfection, such as the quantity of added electrolytic solution, the set electrode voltage or current, etc., are not only dependent on the electrolytic reactor used, such as its reaction volume, its anode and cathode surface, the residence time in the reactor of the water to be disinfected, etc., but in particular also on the composition of the water to be disinfected, particularly its conductivity and redox potential. The usually empirically determined process parameters for a specific water and which in the case of said water lead to a satisfactory disinfecting action, in the case of another water can lead to a very inadequate disinfecting action.

It has in particular been found that the solutions produced according to the prior art by electrochemical activation are generally and in part to a significant extent contaminated with undesired products and frequently according to reaction equation 93) substantially exclusively chlorine gas (Cl₂) is produced, which although desired in standard electrolytic processes, is not desired in electrochemical activation for the production of a disinfectant, because it gives rise to a pungent smell of the electrochemically activated solution. Moreover, as a result of the widely varying composition of the electrochemically activated solution as a function of the aforementioned parameters, no reliable information can be obtained regarding the stability or storage stability of the electrochemically activated solution, so that in practice only an in situ production thereof can be considered. Thus, mainly as a result of poor manipulatability or the only inadequately possible guarantee of an adequate disinfection, the process has not proved commercially successful.

A process for the production of a disinfectant by electrochemical activation is e.g. known from DE 20 2005 008 695 U1.

Therefore the problem of the invention is to so further develop a process for the production of a disinfectant by electrochemical activation (ECA) of water of the aforementioned type that it is possible to ensure a substantially constantly high disinfecting action of the disinfectant or the water to be disinfected and which in particular also satisfies the German Drinking Water Ordnance. It is also directed at a disinfectant produced by means of such a process and the use thereof.

From the process engineering standpoint this problem is solved in the case of a process of the aforementioned type in that the pH-value of the dilute water/electrolytic solution in the anode compartment of the electrolytic reactor is controlled to a value between 2.5 and 3.5. With regards to the actual disinfectant, the invention solves the fundamental problem by means of a disinfectant produced by such a process in the form of an electrochemically activated, anodic, dilute water/electrolytic solution, the pH-value of the disinfectant being between 2.5 and 3.5.

It has surprisingly been found that on setting the pH-value of the dilute water/electrolytic solution, i.e. the water to be disinfected with the electrolyte mixed into the same, such as the sodium or potassium chloride solution, in the electrolytic reactor anode compartment to a value between approximately 2.5 and approximately 3.5, preferably a value between approximately 2.7 and approximately 3.3, particularly a value between approximately 2.8 and approximately 3.2, e.g. to a value between approximately 2.9 and approximately 3.1, as well as to a value of approximately 3.0, not only is there a substantially constant disinfecting action for drinking and service waters with a substantially random composition, but also an adequate depot action, which requires no further disinfection steps and which in particular also persists in the case of sudden loads. The inventive control of the pH-value of the dilute water/electrolytic solution in the electrolytic reactor anode compartment leads to a potential-controlled anodic oxidation (PAO) and in particularly preferred manner there is a redox potential between approximately 1240 and approximately 1360 mV, preferably between approximately 1280 and approximately 1360 mV, particularly between approximately 1320 and approximately 1360 mV. Research has not only shown that the Escherichia coli, Pseudomonas aeruginosa and Enterococcus faecium used as test bacteria in the case of a dilution of the anodic, dilute water/electrolytic solution produced inventively by electrochemical activation under potential-controlled anodic oxidation of approximately 1:400 can be reduced within approximately 30 s by more than four powers of ten, but also that old pipelines with an already optically visible attack of a biofilm or biolawn within two to four weeks are substantially completely freed from said biolawn. Thus, a dilution of approximately 1:400 represents a highly efficient dilution in itself suitable for e.g. disinfecting drinking and service water or swimming pool water. As will be explained hereinafter, it is naturally also possible to use the electrochemically activated, anodic, dilute water/electrolytic solution, e.g. in more concentrated form for numerous further applications. It is naturally also possible in the case of a disinfection of water to add for only a limited time period higher concentrations or lower dilutions of the anodic water/electrolytic solution to the water to be disinfected, so that in the case of calamities occurring in the pipe systems to ensure an effective acute treatment. Such dilutions can then e.g. be between approximately 1:100 and approximately 1:200, the dilution naturally being dependent on the given application.

It has also been found that the setting of the pH-value of the disinfectant in the form of the anodic, dilute water/electrolytic solution in the electrolytic reactor does not bring about in the inventive pH-value range a permanently reduction of the pH-value of the water to be disinfected to said value, which could be undesirable as a result of the relatively acid pH-value for many potential applications of electrochemical activation, e.g. for drinking water. This is on the one hand due to the very low disinfectant quantity necessarily dosed in for disinfecting water (e.g. in a dilution of approximately 1:300 to 1:500), which as a function of the buffer capacity of the water even in the case of very soft waters gives rise to a pH-value reduction of max approximately −0.2. On the other hand after a certain time the pH-value rises again and it is assumed that this can be attributed to the decomposition of the metastable substances, such as ozone, different radicals, etc. (cf. also the above reaction equations) produced in connection with the electrochemical activation. However, as stated, an excellent disinfecting action is still obtained.

However, surprisingly it is also possible to reduce to a minimum the formation of chlorine gas according to reaction equation (3), so that the disinfectant in the form of the electrochemically activated, anodic, dilute water/electrolytic solution at the most only has a very weak chlorine smell, whereas the disinfected product, such as water added to said solution in the appropriate dilution has no chlorine-typical smell. As a result of the potential-controlled anodic oxidation mainly hypochlorites, such as sodium hypochlorite (NaClO) and hypochlorous acid (HOCl), metastable radical compounds and to a lesser extent hydrogen chloride instead of chlorine gas (Cl₂) are produced, i.e. the equilibrium of reaction equation (4) is clearly displaced to the right by the conduction of the process according to the invention. As a result of the spatial separation of the anode compartment, in which the highly disinfection-active, electrochemically activated, dilute water/electrolytic solution is produced, from the cathode compartment, a mixing of the products produced in the anode compartment in connection with the electrochemical activation are prevented from mixing with the products produced in the cathode compartment, so that no substances little or less suitable for disinfecting water are obtained. This can e.g. take place by separating the anode compartment from the cathode compartment of the electrolytic reactor by means of a diaphragm, a membrane or the like, which is electrically conductive, but substantially liquid-tight. In this connection e.g. a diaphragm/membrane of porous zirconium dioxide (ZrO₂) and/or porous aluminium oxide (Al₂O₃) has proved suitable.

In the sense of the invention “control of the pH-value to a value in the range 2.5 to 3.5” is intended to mean that the solution or electrochemically activated, anodic, dilute water/electrolytic solution leaving the electrolytic reactor anode compartment and which is spatially separated from the cathode compartment by an electrically conductive diaphragm/membrane has a pH-value such, i.e. the pH-value control takes place in such a way, that this pH-value has been set in the anode compartment at the end of the reactor. The same applies for the “control of the redox potential of the dilute water/electrolytic solution” to a value between 1240 and 1360 mV, which is to take place in such a way that the solution or electrochemically activated, anodic, dilute water/electrolytic solution leaving the electrolytic reactor anode compartment has a redox potential, i.e. the redox potential control takes place in such a way that this value has been set in the anode compartment at the end of the reactor. The redox potential relates to the normal (NHE) or standard hydrogen electrode (SHE). The term “control” means both a suitable, more or less static presetting of the process parameters, and in particular a dynamic control of the process parameters during reactor operation.

The pH-value of the dilute water/electrolytic solution in the anode compartment can be fundamentally controlled in different ways. Whereas it is e.g. in principle possible for this purpose to add a suitable acid quantity, such as a mineral acid or an organic acid to the water to be disinfected, in addition to the electrolyte, in a preferred variant the pH-value is controlled to the inventive value range without dosing in such an additional acid. As is apparent from the reaction equations taking place at the anode, with the oxidation reactions taking place at the anode frequently protons or H₃O⁺ ions are produced, which consequently reduce the pH-value. It is therefore possible to set the pH-value solely from the reactions taking place at the anode and within the inventive range, a rise in the conversion at the anode obtained during electrochemical activation leads to an increased production of protons and therefore a lower pH-value. The conversion obtained in connection with electrochemical activation in the case of a predetermined reaction geometry can be further increased in that an increased current flow is set between the electrodes or in that a higher voltage is applied to the electrodes (which leads to a higher current flow between the electrodes), in that the residence time of the dilute water/electrolytic solution in the reactor is increased or if there is a continuous or semicontinuous process performance, (in that the volumetric flow of the dilute water/electrolytic solution through the reactor is slowed down), and/or in that more electrolytic solution, e.g. more sodium/potassium chloride solution is dosed in, i.e. an increased educt quantity is used. As stated hereinbefore, the total sodium/potassium chloride concentration should not exceed roughly 20 g/l.

As a result of said dependence of the pH-value of the dilute water/electrolytic solution on the electrolyte conversion in the reactor anode compartment, it is also possible to directly or indirectly measure the pH-value so as in this way to obtain an actual or real value, which can be controlled to the inventive desired value, e.g. by means of conventional control equipment, such as a PID controller. Thus, according to a preferred embodiment of the invention the pH-value of the dilute water/electrolytic solution is directly measured by a pH-meter. Alternatively or additionally the pH-value of the dilute water/electrolytic solution can be indirectly measured via the current flowing between the electrodes (e.g. by means of the voltage necessary for obtaining such a current and which must be applied to the electrodes) and/or by measuring the pH-value of the dilute water/electrolytic solution indirectly via the residence time in—or in the case of a (semi)continuous process performance, via the volumetric flow thereof through the electrolytic reactor.

As has already been indicated, according to a preferred embodiment of the inventive process, the pH-value of the dilute water/electrolytic solution in the anode compartment of the electrolytic reactor is controlled by the controlled addition of a corresponding electrolytic solution quantity, i.e. more electrolytic solution, i.e. more educt is added if the pH-value has to be lowered in order to be controlled in the inventive range, whereas less electrolytic solution, i.e. less educt is added if the pH-value is to be increased in order to be controlled in the inventive range. The dosing in of the corresponding electrolytic solution quantity, e.g. sodium/potassium chloride solution, can e.g. take place by means of a dosing pump, the resulting water/electrolytic solution being preferably very homogeneously mixed prior to entering the electrolytic reactor in order to ensure a homogeneous electrolytic reactor operation. A suitable mixing device has in particular proved to be a ball mixer, in which the dilute water/electrolytic solution is passed through a ball bed.

Alternatively or additionally in a preferred development, the pH-value of the dilute water/electrolytic solution in the anode compartment is controlled by controlling the current flowing between the electrodes, in that e.g. a suitable voltage is applied to the electrolytic reactor electrodes and ensures the current flow necessary for obtaining the inventive pH-value range. It is also alternatively or additionally possible to control the pH-value of the dilute water/electrolytic solution in the anode compartment by controlling the residence time and/or volumetric flow thereof in or through the electrolytic reactor. In each case one or more parameters can be substantially held at or controlled to a constant value.

In order to calibrate an electrolytic reactor with a predetermined reactor geometry as a function of the water to be disinfected, i.e. in order to carry out a setting of the process parameters necessary for the inventive value range for a control of the pH-value of the dilute water/electrolytic solution, according to an advantageous development of the invention for controlling the pH-value of the dilute water/electrolytic solution in the electrolytic reactor anode compartment to a pH-value between 2.5 and 3.5, particularly between 2.7 and 3.3, at a predetermined desired current between the electrodes of the electrolytic reactor, a residence time (Tv) dependent on the composition of the water to be disinfected and/or a volumetric flow (V′) of the dilute water/electrolytic solution in or through the electrolytic reactor dependent on the given composition of the water to be disinfected is determined. In an advantageous embodiment of the invention, the residence time (Tv) and/or volumetric flow (V′) in or through the electrolytic reactor is determined as a function of the conductivity (K) and/or hardness (H) of the water. Since in particular the conductivity of the water has a relatively major influence on the residence time of the dilute water/electrolytic solution in the reactor necessary for achieving the inventive pH-value or in the case of a (semi)continuous conducting of the process, on the corresponding volumetric flow through the reactor, it is possible in this way to initially determine an appropriate residence time/volumetric flow for the water to be disinfected and for determining said dependence a suitable current is applied to the reactor electrodes and can e.g. be empirically determined and is preferably kept constant, in the same way as the quantity of dosed in electrolytic solution. The residence time or volumetric flow, instead of as a function of the conductivity of the water to be disinfected, can be determined as a function of the representative water hardness for the electrical conductivity of the water, i.e. in place of the conductivity of the water proportional to the total concentration of ions contained in the water use is only made of the water hardness, i.e. its calcium and magnesium ion concentration.

In this connection, according to a preferred variant the residence time (Tv) and/or volumetric flow (V′) is determined according to the straight line equation of form

Tv=k₁·K+k₂

and/or

V′=k₃·H+k₄

in which k₁, k₂, k₃ and k₄ are reactor-specific constants. Said straight line equations can e.g. be simply determined in that waters having different conductivity are electrochemically activated at a specific residence time in or with a specific volumetric flow through the reactor and under a specific, particularly constant electrolytic solution inward dosing and for a specific, particularly constant current flow between the electrodes of the electrolytic reactor and the pH-value is controlled to the inventive value range and then the residence times or volumetric flows necessary for the different waters are plotted as a function of the electrical conductivity of the water and the reactor is operated with a corresponding volumetric flow or residence time.

In a further preferred variant of the inventive process, for controlling the pH-value of the dilute water/electrolytic solution in the electrolytic reactor anode compartment to a pH-value between 2.5 and 3.5, particularly between 2.7 and 3.3, for a predetermined residence time (Tv) of the water/electrolytic solution in the electrolytic reactor and/or for a predetermined volumetric flow (V′) of the water/electrolytic solution through said electrolytic reactor, a static desired current (I_{des, stat}) between the electrodes dependent on the water to be disinfected is determined. Here again it is advantageous if the static desired current (I_{des, stat}) is determined as a function of the conductivity (K) and/or hardness (H) of the water. It is preferably once again provided that the static desired current (I_{des, stat}) is determined according to a straight line equation of form:

I_{des,stat}=K₁·K+K₂

and/or

I_{des,stat}=K₃·H+K₄

in which K₁, K₂, K₃ and K₄ are reactor-specific constants.

In order in addition to a more or less static control of the anodic potential of the electrolytic reactor of the aforementioned type to ensure a dynamic control as a function of the optionally time-varying, actual parameters, in a preferred embodiment of the inventive process, for controlling the pH-value of the dilute water/electrolytic solution in the electrolytic reactor anode compartment to a pH-value between 2.5 and 3.5, particularly between 2.7 and 3.3, at a predetermined static desired current (I_{des, stat}) between the electrolytic reactor electrodes, a dynamic desired current (I_{des, dyn}) dependent on the residence time of the water/electrolytic solution in the electrolytic reactor and/or the volumetric flow of the water/electrolytic solution through the electrolytic reactor is determined, so that the control of the reactor for the purpose of maintaining the pH-value of the electrochemically activated, anodic water/electrolytic solution in the range of 3 can take place in situ as a function of the measured actual parameters, such as the residence time or volumetric flow.

The dynamic desired flow (I_{des, dyn}) is preferably also determined according to a straight line equation of form

I_{des,dyn}=K₅·Tv+K₆

and/or

I_{des,dyn}=K₇·V′+K₈

in which K₅, K₆, K₇ and K₈ are reactor-specific constants.

Application takes place to the electrolytic reactor electrodes in the case of such a control, which covers both static setting components dependent on the water used and dynamic control components dependent on the actually measured parameters, preferably application takes place of a total desired current (I_{des, tot}), which is formed from the sum of the static desired current (I_{des, stat}) and dynamic desired current (I_{des, dyn})

I_{des,tot}=I_{des, stat}+I_{des, dyn}.

As has already been stated the dosed in electrolytic solution quantity is kept preferably substantially constant regarding the presetting of the reactor and also during operation, the reactor, e.g. always in its optimum operating state being operable with a specific water volumetric flow (or with a specific water residence time in the reactor) and e.g. in the case of disinfecting water with high demand peak loads and low demand rest periods, said reactor can be switched off every so often (e.g. during rest periods) and a storage means for the produced, electrochemically activated, anodic, dilute water/electrolytic solution can be made available for bridging peak loads.

A particular advantage of the dynamic control component of the inventive control is that in the case where the measured residence time of the dilute water/electrolytic solution and/or the measured volumetric flow thereof in or through the electrolytic reactor drops, with such a measured drop of said residence time and/or said volumetric flow the total current (I_{des, tot}) flowing between the electrodes can also be temporarily reduced due to the dynamic component (I_{des, dyn}). It has been found that particularly in the case of a dynamic operation of the electrochemical activation small gas bubbles can form, such as chlorine gas and oxygen gassed out of the water to be disinfected, so that the conversion obtained during electrochemical activation is impaired and consequently the pH-value rises and also there is a reduction in the volumetric flow through the reactor. To counteract this, it has proved advantageous to have a temporary reduction of the current flowing between the electrolytic reactor electrodes, which is possible through the dynamic component of the desired current. Such a dynamic control or regulation measure can also be appropriate independently of the inventively provided static control of the pH-value of the water/electrolytic solution as a function of the water conductivity or hardness, in order to eliminate a formation of small gas bubbles in the electrolytic reactor attributable to a type of unstable equilibrium and once again return to an operation of the electrochemical activation suitable for a completely satisfactory water disinfection.

According to an advantageous embodiment of the inventive process, the electrolytical solution can be added in the form of a substantially pure alkali metal chloride solution, particularly in the form of a sodium (NaCl) and/or a potassium chloride solution (KCl). In a preferred variant, the electrolytic solution is added in the form of a substantially saturated alkali metal chloride solution. To ensure high reproducibility, the alkali metal chloride solution should be very pure, i.e. it should in particular be substantially free from other halide ions, i.e. those from the group bromide (Br⁻), fluoride (F⁻) and iodide (I⁻), oxohalide ions, such as hypochlorite (ClO⁻), chlorite (ClO₂⁻), chlorate (ClO₃⁻), perchlorate (ClO₄⁻), bromate (BrO₃⁻) etc. It should also be substantially free from heavy metals, particularly from the group antimony (Sb), arsenic (As), lead (Pb), cadmium (Cd), chromium (Cr), nickel (Ni), mercury (Hg), selenium (Se), iron (Fe) and manganese (Mn), as well as preferably substantially no hardening alkaline earth metals, such as in particular calcium (Ca) and magnesium (Mg).

The specific electrical conductivity of the electrolytic solution (prior to the dosing thereof to the water to be electrochemically activated) can preferably be set to a value between approximately 1.5·10⁵ and approximately 3.5·10⁵ μS/cm, particularly between approximately 1.8·10⁵ and approximately 2.8·10⁵ μS/cm, preferably between approximately 2.0·10⁵ and approximately 2.5·10⁵ μS/cm.

The electrolyte concentration, particularly the alkali metal chloride concentration, of the dilute water/electrolyte solution added to the electrolytic reactor (i.e. after dosing the electrolytic solution into the water to be electrochemically activated), particularly the water/alkali metal chloride solution, should, as stated hereinbefore, fundamentally not exceed a value of about 20 g/l. Advantageously the value should be between approximately 0.1 and approximately 10 g/l, particularly between approximately 0.1 and approximately 5 g/l, preferably between approximately 0.1 and approximately 3 g/l (in each case gram per litre electrolyte or alkali metal chloride). Such a concentration has proved suitable for an optimum disinfecting and depot action of the electrochemically activated, dilute water/electrolytic or water/alkali metal chloride solution and also makes it possible to set a favourable pH-value of the electrochemically activated, anodic, dilute water/electrolytic solution at the exit from the electrolytic reactor in the range of around 3 and a redox potential of about 1340 mV vs. SHE (standard hydrogen electrode).

According to a further development of the inventive process, which in itself, i.e. without controlling the pH-value of the dilute water/electrolytic solution in the electrolytic reactor anode compartment, leads to a significant improvement to the electrochemical activation of water, provides for the specific electrical conductivity of the water to be electrochemically activated, prior to the addition of the electrolytic solution, to be set to a value of max 350 μS/cm. It has surprisingly been found that through such a setting of a specific electrical conductivity of the water or untreated water used to a value of max approximately 350 μS/cm, preferably to a value between approximately 0.055 and approximately 150 μS/cm and particularly to a value between approximately 0.055 and approximately 100 μS/cm, prior to the addition of the electrolytic solution (which in any case generally increases by a multiple the conductivity of the water used), an even better reproducibility of the process regarding the disinfecting and depot action of the disinfectant in the form of anodic, electrochemically activated, dilute water/electrolytic solution is ensured and largely independently of the water used. Such a “standardization” of the untreated water used not only permits a particularly easy setting of the process parameters, such as electrode voltage or current, residence time of the dilute water/electrolytic solution in the electrolytic reactor, dosed in electrolytic solution quantity, etc., but also permits in a simple manner a use of waters having a substantially random composition without impairing the disinfectant obtained, so that it is possible to ensure an extremely reliable, potential-controlled, anodic oxidation of the dilute water/electrolytic solution in the electrolytic reactor anode compartment.

Moreover, ions which may be contained in the water to be electrochemically activated, and which during electrochemical activation, even if only in small concentrations, can be transformed into health-hazardous substances, are largely eliminatable. As an example mention is made of bromide ions, which can be oxidized to bromate, as with the ozonization frequently carried out with drinking water treatment, which has a cancerogenic action in higher concentrations. In practice, the process water supplied to the electrolytic reactor upstream of the dosing in of the electrolytic solution, can be investigated and, as a function of the water characteristics, if necessary or constantly deionized or demineralized (as will be explained hereinafter), by means of a preferably continuously operating conductivity measuring cell or electrode with respect to a specific electrical conductivity.

The term “specific electrical conductivity” of the water or the dilute water/electrolytic solution means in the present invention the specific ionic conductivity which is based on the conductivity of the water or water/electrolytic solution as a result of the movable ions dissolved therein.

According to a preferred development the hardness of the water to be electrochemically activated is set, prior to electrolytic solution addition, to a value between approximately 0 and approximately 12° dH, particularly between approximately 0 and approximately 4° dH, preferably between approximately 0 and 2° dH, e.g. between approximately 1 and 2° dH. In this connection “hardness” means the concentration of divalent alkaline earth metal ions, i.e. calcium (Ca), magnesium (Mg), strontium (Sr) and barium (Ba), the two latter ions in practice playing no part. 1° dH corresponds to an alkaline earth metal ion concentration of 0.179 mmole/l, 2° dH to a concentration of 0.358 mmole/1, etc. Such a procedure is particularly appropriate with relatively hard, calcium and/or magnesium-containing waters, in order to increase the electrolytic reactor life or extend its maintenance intervals. However, particularly in the case of very conductive waters, i.e. those with a high total ion concentration, care must be taken to ensure that the water is not merely softened by means of an ion exchanger, because said ion exchanger, in each case replaces a divalent alkaline earth metal ion by two monovalent alkali metal ions and therefore overall further increases the conductivity. It can therefore be appropriate to initially soften the water and then lower the conductivity to a value within the inventive range.

Moreover, particularly in the case of organically burdened or eutrophic waters, it can be advantageous for the total organic carbon (TOC) of the water to be electrochemically activated to be set to a TOC value of max approximately 25 ppb (parts per billion), particularly max approximately 20 ppb, preferably max approximately 15 ppb. Correspondingly with regards to the chemical oxygen demand, this is advantageously set at a COD value of max approximately 7 mg O₂/l, particularly max approximately 5 mg O₂/l, preferably max approximately 4 mg O₂/l.

For setting or lowering the specific electrical conductivity and/or the hardness of the water to be electrochemically activated e.g. membrane processes, such as reverse osmosis, micro-, nano-, ultra-filtration, etc. have proved suitable, but obviously other suitable processes can also be used. For setting or reducing the total organic carbon (TOC) and/or the chemical oxygen demand of the water to be electrochemically activated, use can e.g. be made of oxidation processes, particularly using electromagnetic radiation in the ultraviolet range (UV radiation), or also other known processes.

The control of the electrolyte concentration, particularly the alkali metal chloride concentration, of the dilute water/electrolytic solution, particularly the water/alkali metal chloride solution, added to the electrolytic reactor, preferably takes place by controlling the electrolytic solution quantity added to the water to be electrochemically activated, e.g. using a dosing pump. To ensure a homogeneous concentration distribution, the water to be electrochemically activated is appropriately intimately mixed after dosing in the electrolytic solution.

According to a preferred embodiment the control of the alkali metal chloride concentration of the dilute water/alkali metal chloride solution added to the electrolytic reactor can be carried out as a function of the corresponding specific electrical conductivity of the dilute water/alkali metal chloride solution added to the electrolytic reactor, the dependence of the alkali metal chloride concentration on the specific electrical conductivity of the dilute water/alkali metal chloride solution added to the electrolytic reactor being predetermined for the water to be electrochemically activated and of which use is made. After determining this dependence in the form of a calibration curve, it is then only necessary to measure the representative conductivity for the alkali metal chloride concentration of the dilute water/alkali metal chloride solution and convert it by means of the calibration curve into the alkali metal chloride concentration. Knowing the concentration of the alkali metal chloride solution available it is consequently possible to dose in the in each case necessary quantity for obtaining the desired concentration, which appropriately takes place by means of an electronic data processing unit, which is on the one hand connected to a conductivity measuring cell or electrode and on the other to a corresponding dosing member.

It has proved particularly appropriate if the dependence of the alkali metal chloride concentration on the specific electrical conductivity of the dilute water/alkali metal chloride solution added to the electrolytic reactor is determined according to a calibration line of form

Kₜₒₜ=K_{w}+dK/d[MeCl]·[MeCl]

in which Kₜₒₜ is the specific electrical conductivity of the dilute water/alkali metal chloride solution added to the electrolytic reactor, K_{w} the specific conductivity of the particular water to be disinfected (directly prior to adding the electrolytic solution, preferably max 350 μS/cm), [MeCl] the alkali metal chloride concentration of the dilute water/alkali metal chloride solution added to the electrolytic reactor and dK/d[NaMe] the water-specific gradient of the calibration line, i.e. the constant dK/d[MeCl] is dependent on the contents of the water used and whose specific electrical conductivity at the time of dosing in the electrolytic solution, as stated, can already be set to a value of max approximately 350 μS/cm. For carrying out such a calibration it is e.g. possible for a specific, known concentration of the stocked alkali metal solution to be dosed in to the water at a known, e.g. measured conductivity K_{w} of the water to be electrochemically activated to measure the total conductivity Kₜₒₜ of the dilute water/alkali metal chloride solution at different quantities of added alkali metal chloride solution. If these measured values for the total conductivity Kₜₒₜ of the dilute water/alkali metal chloride solution are plotted on the ordinate compared with the alkali metal chloride concentration [MeCl] of the dilute water/alkali metal chloride solution on the abscissa, the calibration line is obtained, where the factor dK/d[MeCl] represents the gradient of the line and the value K_{w} of the ordinate intersection of said line. As stated, [MeCl] is preferably e.g. [NaCl] and/or [KCl].

As has already been stated, not only with regards to the production of the inventive disinfectant, but also in the case of a disinfection of water by means of such a disinfectant, it can be advantageous to exclusively use the electrochemically activated, dilute water/electrolytic solution produced in the anode compartment and to discard the dilute water/electrolytic solution produced in the cathode compartment and which is less suitable for disinfection.

For the disinfection of water or also random other media, it can be appropriate if the disinfectant is used in substantially pure form or in the form of a dilution of up to 1:500, particularly up to 1:400 parts of a diluent, particularly water.

Apart from the production of the disinfectant in the pure state, the inventive process can also be used for disinfecting water, such as drinking and service water, rain water, swimming pool water, industrial water and waste water, etc. In this connection it is e.g. favourable from the process engineering standpoint if a partial flow is branched off from the water to be disinfected, said partial flow is electrochemically activated and at least (or exclusively) the partial flow electrochemically activated in the anode compartment is added as disinfectant to the water to be disinfected and said disinfectant, as stated and as a function of the intended use is added again in an appropriate dilution to the water to be disinfected.

Finally the inventive process is particularly suitable for continuous or semicontinuous performance, a partial flow of the water to be disinfected or the anodic, dilute water/electrolytic solution inventively electrochemically activated for producing the disinfectant is passed (semi)continuously through the electrolytic reactor.

The invention also relates to a disinfectant in the form of an electrochemically activated, anodic, dilute water/electrolyte solution (anolyte), produced in the inventive manner, whose pH-value is between approximately 2.5 and approximately 3.5, preferably between approximately 2.7 and approximately 3.3, particularly between approximately 2.8 and approximately 3.2 and whose redox potential in an advantageous variant is between approximately 1240 and approximately 1360 mV, preferably between approximately 1280 and approximately 1360 mV, particularly between approximately 1320 and approximately 1360 mV.

The electrochemically activated, anodic, dilute water/electrolytic solution produced according to the inventive process can be used as a disinfectant, e.g. wherever a completely satisfactory disinfection of water, particularly complying with the Drinking Water Ordnance is needed and also for disinfecting the communal water supply or the water supply of hospitals, schools, care homes, in trading premises, hotels or other gastronomical enterprises and sports associations (e.g. for dosing in water for the sanitary installations), stations, airports, industrial kitchens, for disinfecting swimming pool or rain water (e.g. for adding in the case of rain water treatment) or for adding to water storage tanks of random types, for desalination plants, such as sea water desalination plants on ships or on land, for preventing the carrying of bacteria into the water of textile washing machines, for the rapid decolorizing of dye works waste waters, for random industrial (waste) waters, such as for admixing to cooling water (e.g. for turning, milling, drilling, cutting or other machine tools), for air conditioning and air humidifying systems, for osmosis plants, as an additive to the water for mixing concrete and cement, as an additive to the water in the production of electronic components and circuits, as an additive to the water for cut flowers, for dosing into the drinking and waste water of animal keeping enterprises and abattoirs or for the disinfection of the equipment used in this connection, such as incubators, milking machines, etc. In all cases for acute disinfection or during normal operation a reliable disinfection is obtained and the undesired formation of algae and/or sludge is prevented. The disinfectant can either be used in substantially pure form or, particularly in the case of water treatment, in the form of a dilution of up to approximately 1:500, preferably up to approximately 1:400 parts of a diluent, such as water and in the case of water treatment, e.g. a dilution in the range of approximately 1:400 has in many cases proved appropriate.

The disinfectant in the form of an inventive electrochemically activated, anodic water/electrolytic solution can also be used, e.g. in pure form or particularly with a suitable dilution, for disinfecting foods, such as cereals or flour, spices, fruit, vegetables, ice cream and ice used as a coolant or refrigerant, e.g. in connection with the storage of fish, meat and seafood in connection with transportation and sales, animal products, etc., a completely satisfactory killing of bacteria, such as putrefactive bacteria, etc. is obtained and therefore a longer storage stability is brought about with very good health compatibility characteristics.

The inventively produced disinfectant can also be used for disinfecting seed, and can e.g. be used as an ensilaging and preserving agent on storing seed and cereals in silos.

Another preferred use of such a disinfectant involves the disinfection of packing containers and packs, particularly for hygienic products, such as foods, pharmaceuticals, sterile articles (such as syringes, surgical instruments, etc.), and the like.

In addition, it is advantageous to use such a disinfection for reaction media for carrying out solvent and emulsion polymerizations, the use of the emulsifiers necessary being reduced and the polymerization rate can be surprisingly increased, as has been shown in an experiment in connection with the production of divinyl styrene rubber.

A further preferred use of such a disinfectant is as an additive for in particular water-soluble paints, varnishes, lacquers and pigments, which can give a biocidal effect, as well as an additive for coolants and lubricants, e.g. for industrial cooling circuits or for industrial lubricants based on water, oil or grease.

Finally, such a disinfectant can also be used as an additive for fuels and propellants, such as heating oil, petrol/gasoline, paraffin/kerosene, etc.

In all cases the disinfectant in the form of an anodic water/electrolytic solution electrochemically activated according to the invention, in the case of suitable storage (particularly substantially under an oxygen seal) can be easily stocked for up to about six months.

Hereinafter the inventive process is explained in greater detail relative to embodiments of a process for the treatment or disinfection of drinking water with reference to the drawings. It is pointed out that the production of electrochemically activated, anodic, dilute water/electrolytic solution in pure or otherwise dilute form can take place in an identical electrolytic reactor. In the drawings show:

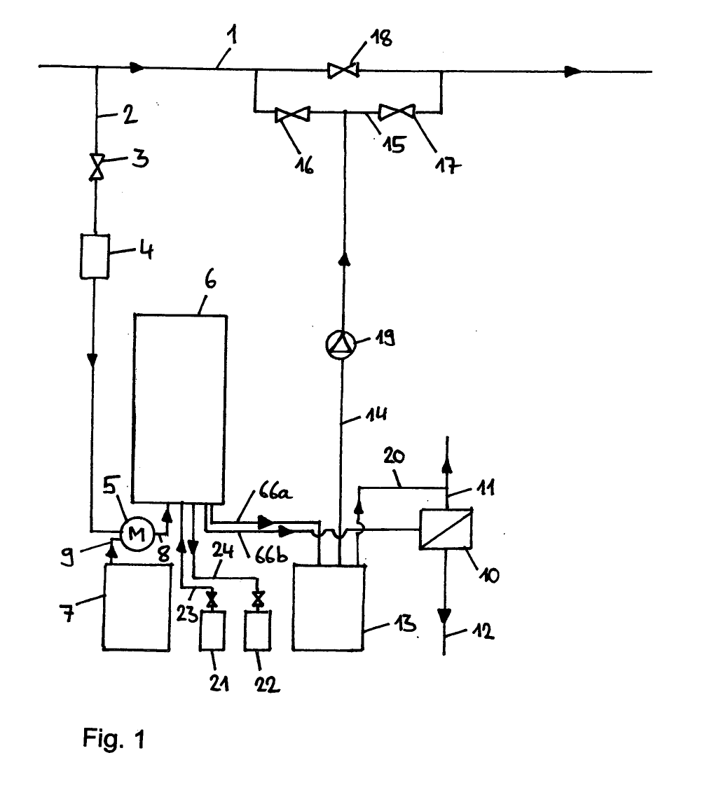

FIG. 1 An inventive flow chart of a first embodiment of an inventive process for disinfecting water by electrochemical activation (ECA).

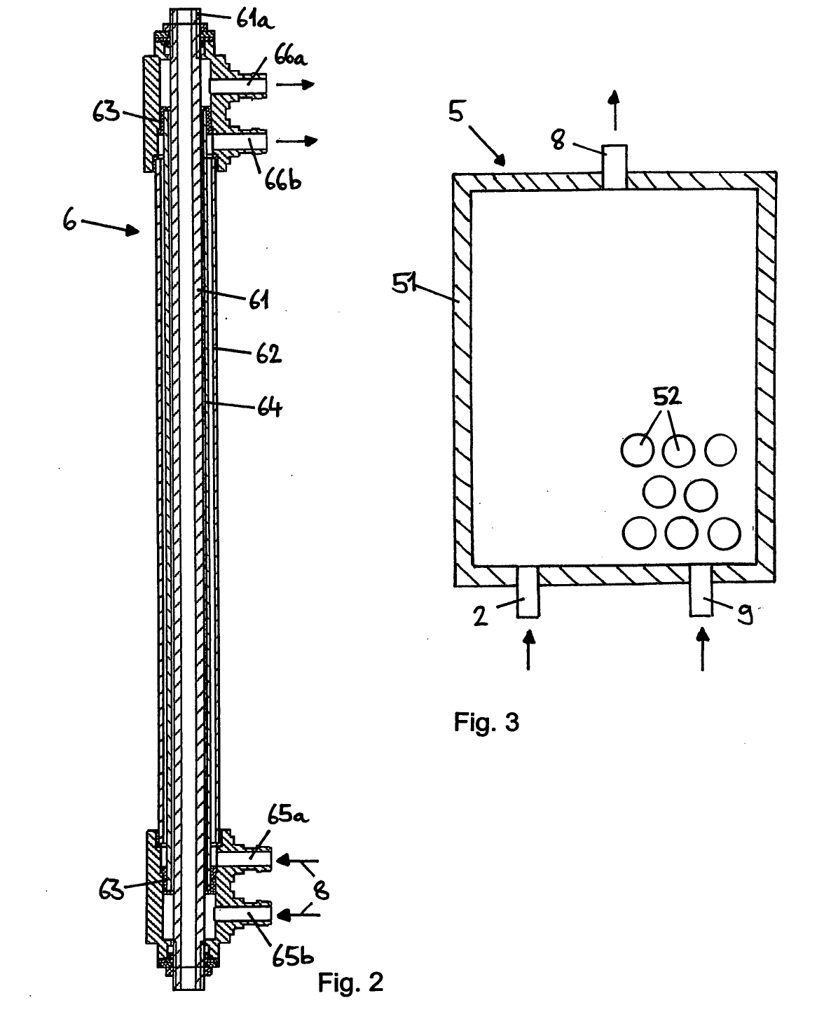

FIG. 2 A sectional detail view of the electrolytic reactor according to FIG. 1.

FIG. 3 A sectional detail view of the mixer according to FIG. 1.

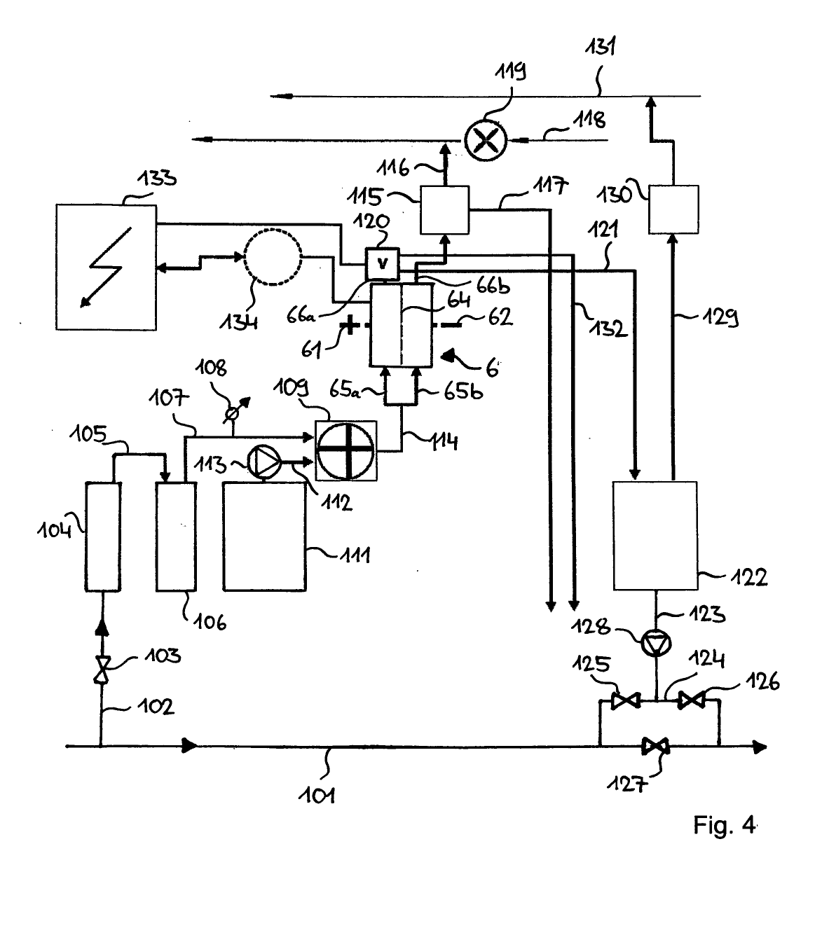

FIG. 4 A diagrammatic flow chart of a second embodiment of an inventive process for disinfecting water by electrochemical activation (ECA), which differs from the embodiment according to FIG. 1 particularly through the use of a clean water plant upstream of the electrolytic reactor.

The apparatus for disinfecting water by electrochemical activation (ECA) under potential-controlled, anodic oxidation (PAO) for the continuous or semicontinuous performance of an inventive process diagrammatically illustrated in FIG. 1, comprises a main water pipe 1, in which is conveyed the water to be disinfected. The main water pipe 1 can e.g. be formed by a supply pipe for the water supply of a hospital, a trading enterprise, a hotel or some other gastronomic enterprise, as well as by the circulation pipe of a swimming pool or the like. To the main water pipe 1 is connected a branch pipe 2, which is equipped with a valve 3, particularly in the form of a control valve, as well as with a filter 4, particularly in the form of a fine filter with a hole width of e.g. approximately 80 to 100 μm and issues by means of a mixer 5 explained in greater detail relative to FIG. 3 into an electrolytic reactor 6 described in greater detail hereinafter relative to FIG. 2. Thus, by means of branch pipe 2 a partial flow of the water carried in the main water pipe 1 controllable by means of a control valve 3 can be transferred into the electrolytic reactor 6 and e.g. a partial flow of the water in the main water pipe 1 is branched off via branch pipe 2 in a quantity of about 1/200.

Mixer 5 is on the feed side connected to the branch pipe 2 and also to a storage tank 7 for receiving an electrolytic solution, here e.g. a substantially saturated sodium chloride solution, which are homogeneously mixed together in mixer 5 and passed by means of a common, outflow-side pipe 8 of mixer 5 into electrolytic reactor 6. The pipe 9 leading from storage tank 7 into mixer 5 is equipped with a dosing pump not shown in FIG. 1 in order to add a clearly defined electrolytic solution quantity to the water carried in branch pipe 2. As is particularly apparent from FIG. 3, in the present embodiment the mixer 5 is formed by a ball mixer, which ensures a constant, uniform thorough mixing of the water with the electrolytic solution. It essentially comprises a roughly cylindrical container 51, to whose opposing ends are connected the inflows 2, 9 or outflow 8 and in which is placed a bed of balls 52, indicated in exemplified manner in FIG. 3, or some other bulk material, through which the water and electrolytic solution flow, the balls 52 being made to vibrate and thereby ensuring a very homogeneous thorough mixing of the water with the electrolytic solution added thereto.

As can in particular be gathered from FIG. 2, the electrolytic reactor 6 comprises an anode 61, which in the present embodiment, e.g. is constituted by a hollow titanium tube coated with catalytically active ruthenium dioxide (RuO₂) and to which can be terminally connected by an external thread 61 the positive pole of a not shown voltage source. Alternatively or additionally to ruthenium oxide it is e.g. also possible to use a coating based on iridium dioxide (IrO₂) or a mixture of both (RuO₂/IrO₂) or other oxides, such as titanium dioxide (TiO₂), lead oxide (PbO₂) and/or manganese dioxide (MnO₂). Electrolytic reactor 6 also comprises a cathode 62, which is appropriately made from high grade steel or other materials, such as nickel (Ni), platinum (Pt), etc. and which in the present embodiment is also formed by a hollow tube within which is coaxially placed the anode 61. Cathode 62 is connectable by means of not shown terminals externally embracing the same to the negative pole of the not shown voltage source. Coaxial to anode 61 and cathode 62 and between the same is provided a tubular diaphragm 64 sealed by sealing rings 63 and which subdivides the annular reaction chamber between anode 61 and cathode 62 into an anode compartment and a cathode compartment. Diaphragm 64 prevents mixing of the liquid in the anode compartment and cathode compartment, but still permits a current flow, which does not provide a high resistance to the migration of ions. In the present embodiment the diaphragm 64 is made from e.g. electrically or ionically conductive, but substantially liquid-tight, porous zirconium dioxide (ZrO₂). Other materials with a relatively low resistance, such as aluminium oxide (Al₂O₃), ion exchange membranes, particularly those based on plastic, etc., can also be used.

Electrolytic reactor 6 also has two inlets 65a, 65b by means of which the water/electrolytic solution passing out of the mixer 5 by pipe 8 is fed into the reaction chamber of reactor 6, i.e. into its anode compartment and into its cathode compartment spatially separated therefrom by diaphragm 64. For this purpose is provided an e.g. T-shaped branch, which is not shown in FIG. 1. As can in particular be gathered from FIGS. 3 and 1, the electrolytic reactor 6 also has two outlets 66a, 66b by means of which the water/electrolytic solution, following chemical activation in reactor 6 can be removed from the latter. Whereas outlet 66a is used for removing the electrochemically activated water/electrolytic solution from the anode compartment of reactor 6, i.e. for removing the so-called anolyte, outlet 66b is used for removing from the cathode compartment, i.e. for removing the so-called catholyte. On starting up the electrolytic reactor 6, for a certain time period it is also possible to discard the “anolyte”, i.e. the electrochemically activated, anodic water/electrolytic solution in order to exclude initial quality deteriorations, for as long as the electrolytic reactor 6 has not reached its desired operating state.

Hereinafter are given the geometrical dimensions of the electrolytic reactor 6 used in list form:

cathode compartment length: 18.5 cm;
cathode compartment volume: 10 ml;
cathode surface area: 92.4 cm²;
anode compartment length: 21.0 cm;
anode compartment volume: 7 ml;
anode surface area: 52.7 cm²;
distance between cathode and anode: approx. 3 mm (including diaphragm).

Electrolytic reactor 6 is e.g. operated with a water throughput of 60 to 140 l/h, but obviously higher throughputs are possible, in that use is made of larger reactors and/or several parallel-connected reactors. The electrolytic reactor 6 is preferably always operated under full load and if necessary can be disconnected and peak loads can be absorbed by means of a subsequently described storage tank for the electrochemically activated, anodic, dilute water/electrolytic solution.

As can be gathered from FIG. 1, the outlet 66b from the cathode compartment of electrolytic reactor 6 issues into a gas separator 10, from which the spent gas is removed by means of an optionally provided spent gas line 11, whereas the actual catholyte, i.e. the water/electrolytic solution removed from the cathode compartment of the electrolytic reactor 6 is removed via a pipe 12, e.g. into the sewers of a communal waste water system. The outlet 66a from the anode compartment of electrolytic reactor 6 issues into a storage tank 13 from which the anolyte can be added via a pipe 14 to the main water pipe 1, which in the present embodiment takes place by means of a bypass pipe 15, which can be controlled up and down using a control valve 16, 17 in each case positioned downstream or upstream of the connection point of pipe 14 to bypass pipe 15. Another control valve 18 is placed in the section of main water pipe 1 bridged by the bypass pipe 15. In the pipe 14 connecting the storage tank 13 to bypass pipe 15 of main water pipe 1 is provided a dosing pump 19, which is used for the controlled dosing in of anolyte from storage tank 13 into main water pipe 1. A spent gas line 20 issues from storage tank 20 into spent gas line 11 from gas separator 10. The function of bypass pipe 15 to which the disinfectant is added consists in normal operation of passing all the water in the main water pipe 1 via bypass pipe 15 and supplying disinfectant thereto. For maintenance and installation purposes the bypass pipe 15 can be separated via valves 16, 17 from the main water pipe 1.

Electrolytic reactor 6 is also equipped with a controllable voltage source not shown in FIG. 1 in order between anode 61 and cathode 62 (FIG. 2) to control the desired current flow measured by a not shown ammeter. It also has a not shown pH-meter e.g. located in the anolyte outlet 66a, which can alternatively be provided e.g. in storage tank 13. A not shown, controllable pump integrated into reactor 6 is used for the controllable delivery of dilute water/electrolytic solution through the electrolytic reactor, the pump controlling the volume flow and therefore the residence time of the water/electrolytic solution in reactor 6. An also not shown control device, e.g. in the form of an electronic data processing unit, is set up for controlling said parameters in such a way that the anolyte passing out of the anode compartment of reactor 2 via outlet 66a has a pH-value between 2.5 and 3.5, preferably approximately 3.0, which can e.g. be brought about using PID controllers.

For cleaning the electrolytic reactor 6 is also provided a storage unit 21 for receiving cleaning liquid, e.g. acetic acid or the like and optionally a storage unit 22 for receiving the spent cleaning liquid, and a supply line 23 leading from storage unit 21 into reactor 6 can be optionally coupled to the inlets 65a, 65b of reactor 6 (cf. FIG. 2) and an outgoing line 24 leading from reactor 6 into storage unit 22 can if need be coupled with the outlets 66a, 66b of reactor 6 (cf. FIG. 2), so that said reactor 6, i.e. both its cathode compartment and in particular its anode compartment can be rinsed. Alternatively the cleaning solution, particularly in the case of acetic acid, can also be directly fed into an e.g. communal waste water or sewage system.

To increase the service life of the electrolytic reactor 6 or extend its maintenance intervals, upstream thereof can be provided a softener not shown in FIG. 1, which keeps the hardness of the water, e.g. at a value of max 4° (cf. in this connection the subsequently described embodiment according to FIG. 4).

The operation of the apparatus for disinfecting water by electrochemical activation (ECA) using potential-controlled anodic oxidation (PAO) is briefly described hereinafter.

As a function of the electrical conductivity or, in the present embodiment, as a function of the hardness of the water to be disinfected which represents the same and which as a rule has a pH-value in the neutral range, e.g. approximately 6 to 8, the electrolytic reactor 6 undergoes calibration so that, for obtaining a pH-value of approximately 3 in the anode compartment of reactor 6, suitable desired values of the current flowing between the electrodes and the volumetric flow through the reactor or the residence time of the water/electrolytic solution in said reactor 6, particularly in its anode compartment in which is produced the anolyte active in disinfecting the water is obtained. With increasing hardness or electrical conductivity of the water to be disinfected it is necessary to have a higher current and/or a lower volumetric flow or longer residence time, in order to obtain a conversion of the dilute water/electrolytic solution in connection with its electrochemical activation in order to set a pH-value of approximately 3. For calibration initially a volumetric flow through the reactor 6 is set and this roughly corresponds to the preset details regarding the necessary volumetric flow through the reactor 6 or more precisely the volumetric flow supplied via branch pipe 2 to reactor 6, here e.g. approximately 1/200 of the water flow in the main water pipe 1, which is mainly based on the quantity delivered in the main water pipe 1 of anolyte returned by means of the pipe 14 into main water pipe 1 (here e.g. approximately 1/400, of the water flow in main water pipe 1, whereas approximately 1/400 of this flow is discarded in catholyte form). In addition, a flow is set, which results from suitable dosing in of electrolytic solution from storage tank 7 gives a pH-value of roughly 3 for the dilute water/electrolytic solution with regards to electrochemical activation. Following the calibration for different waters with different hardness levels, in the present embodiment the following calibration lines are obtained for the static desired current (I_{des, stat}) or desired volumetric flow through the reactor 6 (V′_{des}):

I_{des,stat}=0.418 A·hardness [°]+0.953 A; (I)

V′_{des}=0.95 l/h·hardness [°]+43.80 l/h. (II)

After establishing the aforementioned calibration lines, the current between anode 61 and cathode 62 of electrolytic reactor 6 is set as a function of the hardness of the water to be disinfected at the corresponding desired value. The same applies for the volumetric flow through reactor 6 of the dilute water/electrolytic solution.

During operation the pH-value of the anolyte used for disinfecting the water is always controlled in such a way that the anolyte pH-value is in the range of about 3, which can in particular take place by additional control of the dynamic component (I_{des, dyn}) of the total desired current (I_{des, tot}) applied to the electrodes 61, 62 of electrolytic reactor 6, whilst taking account of the actually measured volumetric flow (V′) through reactor 6:

I_{des,tot}=I_{des, stat}+I_{des, dyn} (III)

in which I_{des, dyn}=K₇·V′+K₈. As a function of the measured pH-value the current applied to the electrodes 61, 62 is increased if the pH-value rises above 3 (i.e. if the measured volumetric flow V′ increases or if the conversion obtained in connection with electrochemical activation drops), whereas the current is reduced if the pH-value drops below 3 (i.e. if the measured volumetric flow V′, e.g. due to the formation of small gas bubbles in the reaction compartment, decreases or if the conversion obtained during electrochemical activation increases) and/or the volumetric flow V′ through the reactor is reduced if the pH-value rises above 3, whereas the volumetric flow is increased if the pH-value drops below 3. Whilst the quantity of dosed in electrolytic solution is preferably kept substantially constant, alternatively or additionally more electrolytic solution can be dosed in from storage tank 7 if the pH-value rises above 3 (i.e. if the conversion obtained during electrochemical activation decreases), whereas less electrolytic solution is dosed in if the pH-value drops below 3 (i.e. if the conversion obtained during electrochemical activation rises). According to the invention it is particularly also possible to keep constant two of the three aforementioned parameters namely current (i.e. electric current between the electrodes of reactor 6), volumetric flow through the reactor 6 (i.e. volumetric flow of dilute water/electrolytic solution) and dosed in electrolytic solution quantity and to keep the pH-value in the inventive range solely by controlling the third parameter. The redox potential, which in the case of the inventive control of the pH-value is set at a level of approximately 3, is preferably roughly constantly 1340 mV±20 mV.

The disinfecting liquid, which is buffer stored in the storage tank 13 in the form of an anolyte and obtained as a result of the inventively controlled electrochemical activation in the form of a potential-controlled anodic oxidation, is added to the main water pipe 1 by means of dosing pump 19, particularly in a proportion of approximately 1:400, so as to ensure a reliable disinfection of all the water carried therein. As the electrochemically activated anolyte, as stated, is in a metastable state, with regards to the largely unprotected and possibly warm storage it should be stored in the storage tank 13 with a relatively large free surface of the liquid level in said tank 13 for a maximum of about 14 days, preferably a maximum of about 48 hours, prior to its addition to the water for disinfecting purposes. However, as stated hereinbefore, it is also possible to store the disinfectant produced in the aforementioned manner for up to about six months, but it is necessary to ensure a very gas-tight seal of corresponding storage tanks and preferably a very low temperature, e.g. down to approximately 8° C.

FIG. 4 is a process diagram of a further apparatus for the continuous or semicontinuous performance of an inventive process for disinfecting water by electrochemical activation (ECA). The apparatus once again comprises a main water pipe 101 which carries the water to be disinfected, e.g. in the form of a supply pipe for the water supply of a hospital, trading enterprise, hotel or other gastronomic enterprise, the circulating pipe of a swimming pool or the like. To the main water pipe 101 is connected a branch pipe 102, which is equipped with a valve 103, such as a control valve and also can have a not shown filter, particularly in fine filter form.

Downstream of valve 103 branch pipe 102 issues into a softener 104, which can e.g. be equipped with a suitable ion exchange resin and which replaces the divalent hardening calcium and magnesium ions in the water by monovalent ions, such as e.g. sodium. To increase the life of the electrolytic reactor 6 or increase its maintenance intervals, softener 104 keeps the hardness of the water e.g. at a value of max 4° dH (corresponding to an alkaline earth metal ion concentration of 0.716 mmole/l), preferably max 2° dH (corresponding to an alkaline earth metal ion concentration of 0.358 mmole/l). The outflow 105 of softener 104 issues into a device 106 for reducing the specific electrical or ionic conductivity of the water and which can in particular be formed by a membrane plant, such as a reverse osmosis plant or a micro-, nano- or ultra-filtration plant and keeps the specific electrical conductivity of the water at a value of max 350 μS/cm, particularly max 150 μS/cm, preferably max 100 μS/cm. The outflow 107 of membrane plant 106 contains a conductivity measuring device 108, such as a conductivity measuring cell, electrode or the like, for monitoring the maintenance of the in each case desired value for the specific electricity conductivity of the water.

Particularly if the water to be disinfected has a relatively high total organic content, measures are provided for reducing the water carbon content. For this purpose can be provided a not shown UV oxidation plant upstream of the mixer 109, which reduces the total organic content (TOC) and/or chemical oxygen demand (COD) to a value of max 25 ppb, particularly max 20 ppb or a value of max 7 mg O₂/l, particularly max 5 mg O₂/l. For measuring and/or controlling the TOC or COD value, or other group parameters for determining the organic carbon contained in the water, such as the dissolved organic carbon (DOC), use can be made of prior art devices.

It is also conceivable in conjunction with the softener 104 and membrane plant 106 to pass the partial water flow to be electrochemically activated and branched off via branch pipe 102 from the main water pipe 101 when this is necessary through softener 104, membrane plant 106 or the UV oxidation plant, namely on exceeding the given limit value and to otherwise bridge the plant by a not shown bypass pipe.

The outlet 107 of membrane plant 106 leads into a mixer 109 issuing into an electrolytic reactor 6. In the present case the latter corresponds to the embodiment of FIG. 2. Branch pipe 102 is thus able to transfer a softened, deionized partial flow of the water conveyed in the main water pipe 101 and controllable by means of control valve 103 into the electrolytic reactor 6 and e.g. a partial flow of the water carried in the main water pipe 101 and having an order of magnitude of 1/200 is branched off via branch pipe 102. On the inlet side the mixer 109 is connected, as stated, to the outlet 107 of membrane plant 106 and also to a storage tank 111 for receiving an electrolytic solution, particularly in the form of a substantially saturated alkali metal chloride solution, in the present case a sodium chloride solution, which are intimately homogeneously mixed in mixer 109 and pass via a common, outlet-side pipe 114 of mixer 109 into electrolytic reactor 6. The pipe 102 leading from storage tank 111 into mixer 109 is also equipped with a dosing pump, in order to add to the water to be electrochemically activated a clearly defined sodium chloride solution quantity. The mixer 109 can e.g. be formed by a ball mixer according to FIG. 3. Once again the electrolytic reactor 6 is e.g. operated with a water throughput of 60 to 140 l/h and for the reasons given in connection with FIG. 1 it is preferably always operated under full load and if necessary can be switched off.

As can also be gathered from FIG. 4, the outlet 66b from the cathode compartment of electrolytic reactor 6 issues into a gas separator 115, from which the spent gas, particularly hydrogen (H₂), is led off via an optionally provided spent gas line 116, whereas the actual catholyte, i.e. the dilute water/electrolytic solution removed from the cathode compartment of electrolytic reactor 6 is removed via a line 117, e.g. into the sewer of a communal waste water or sewage system. The spent gas line 116 in the present embodiment issues into a spent air line 118 fed with dilution air and which is equipped with an explosion-protected low pressure fan 119.

The outlet 66a from the anode compartment of electrolytic reactor 6 issues via a control valve 120 and a line 121 into a storage tank 122 from which the anolyte can be added via a line 123 to the main water pipe 101. In the present embodiment this takes place by means of a bypass pipe, which can be controlled by a connection point of line 123 located upstream or downstream into the bypass pipe 124 by means of in each case a control valve 125, 126. A further control valve 127 is located in the section of the main water pipe 101 bridged by the bypass pipe 124. In the line 123 connecting the storage tank 122 to the bypass pipe 124 of main water pipe 101 is provided a dosing pump 128, which is used for the controlled dosing in of the anolyte from storage tank 122 into main water pipe 101. A spent gas line 129, particularly for chlorine gas (Cl₂) optionally released in the anolyte, issues from the gas chamber of storage tank 122 into a gas separator 130, whose gas chamber is in turn connected to a chlorine gas removal line. Liquids separated in the gas separator, such as e.g. condensed out water, can also be removed into e.g. the sewer of a communal sewage system (not shown). The function of the bypass pipe 124, which adds the dilute water/sodium chloride solution electrochemically activated in the anode compartment of electrolytic reactor 6 is that in normal operation all the water flow in the main water pipe 101 is led via bypass pipe 124 and can be supplied with the disinfectant. For maintenance and installation purposes the bypass pipe 124 can still be separated from main water pipe 1 via valves 125, 126.

On starting up the electrolytic reactor 110 for a certain time period it is also possible to discard the “anolyte”, i.e. the electrochemically activated, anodic water/electrolytic solution, in order to exclude initial quality deteriorations until the electrolytic reactor 110 arrives at its desired operating state. For this purpose a further line 132 passes from valve 120 parallel to the line 121 leading into storage tank 122 and which e.g. leads into the sewer of a communal sewage system in order to be also able to discard the anolyte, as a function of the switching position of valve 120.

The apparatus of FIG. 4 also comprises a control unit 133, e.g. in the form of an electronic data processing unit, which is on the one hand connected to control valve 120, so that the line 121 or line 132 can when necessary be controlled up and down, but is also connected via a potential control 134 to electrolytic reactor 6, in order to control the desired current flow measured e.g. by a not shown ammeter in the electrolytic reactor 6 between anode 61 and cathode 62 (FIG. 2). For the aforementioned reasons this takes place in such a way that in the electrochemically activated, anodic, dilute water/electrolytic solution a pH-value of approximately 3 is set and also there is a redox potential of approximately 1340 mV, which as a result of the inventive setting of a specific electrical conductivity of the untreated water of max 350 μS/cm so that in simple manner this is possible for virtually all waters having random contents. To this end the line 166a leading from the anode compartment of electrolytic reactor 6 is provided with a not shown pH-meter and preferably also a further conductivity measuring cell or electrode (not shown), which make it possible for the control unit to control the sodium chloride solution quantity added to the untreated water via pump 113 in such a way that the desired process parameters are obtained. The total sodium chloride concentration in the feed inlet 114 of electrolytic reactor 6 should still not exceed roughly 20 g/l, preferably roughly 10 g/l. Control unit 133 can also control a not shown controllable pump integrated into reactor 6 for the controllable delivery of the water/electrolytic solution through electrolytic reactor 6 and consequently by means of the pump it is possible to adjust the volume flow or residence time of the dilute water/electrolytic solution through or in reactor 6.

Hereinafter a brief description is given of the operation of the apparatus for disinfecting water by electrochemical activation (ECA) under potential-controlled anodic oxidation (PAO).

The water branched off from the main water pipe 101 via branch pipe 102, e.g. a volume proportion of approximately 1:200 of the water carried in main water pipe 101, is initially softened in softener 104, e.g. to a hardness of approximately 1° dH (corresponding to an alkaline earth metal ion concentration of calcium and magnesium of 0.179 mmole), after which in membrane plant 106 its specific electrical conductivity is lowered to a value of e.g. approximately 50 μS/cm. In the case of a relatively high organic carbon content of the water, e.g. higher than about 25 ppb, this can be further decreased, e.g. by oxidative degradation.

With such a softened, deionized and optionally oxidatively treated water a calibration is made in order to obtain a correlation between the quantity of sodium chloride solution to be added to the water by means of dosing pump 113 and the total specific electrical conductivity obtained of the resulting dilute water/sodium chloride solution. This dependence of the sodium chloride concentration on the specific electrical conductivity of the dilute water/sodium chloride solution added to the electrolytic reactor is determined in particular according to a calibration line of form

Kₜₒₜ=K_{w}+dK/d[NaCl]·[NaCl]

in which Kₜₒₜ is the specific electrical conductivity of the dilute water/electrolytic solution added to the electrolytic reactor, K_{w} the specific conductivity of the water to be disinfected used (directly prior to the addition of the electrolytic solution, i.e. in the present case with a specific electrical conductivity of approximately 50 μS/cm), [NaCl] the sodium chloride concentration of the dilute water/sodium chloride solution used and dK/d[NaCl] the water-specific calibration line gradient.

Then the electrolytic reactor 6 for the water to be disinfected can undergo calibration so that for obtaining a pH-value of approximately 3 in the anode compartment of the reactor 6 suitable desired values of the current flowing between the electrodes 61, 62 and the volumetric flow through the reactor 6 or the residence time of the dilute water/electrolytic solution in reactor 6, particularly in its anode compartment in which is produced the anolyte active in disinfecting the water are obtained. With rising electrical conductivity of the water to be disinfected a higher current and/or a lower volumetric flow (or a higher residence time) is needed, in order to obtain a conversion of the dilute water/electrolytic solution in conjunction with its electrochemical activation for setting a pH-value of approximately 3.

During operation the pH-value of the anolyte used for disinfecting the water is constantly controlled in such a way that the anolyte pH-value is approximately 3, which more particularly takes place through a corresponding control of the electrical current applied to the electrodes 61, 62 of electrolytic reactor 6 and whilst taking account of the volumetric flow of the water/sodium chloride solution through the reactor 6 and/or by corresponding dosing in of the sodium chloride solution by means of dosing pump 113. The redox potential, which is set at a value of approximately 3 during a control of the pH-value is preferably approximately constantly 130 mV±20 mV vs. SHE.

The disinfecting liquid, which is intermediately stored in anolyte form in the storage tank 122 obtained in this way by inventively controlled electrochemical activation in the form of a potential-controlled anodic oxidation is added to the main water pipe 101 by means of dosing pump 128, e.g. in a proportion of approximately 1:400, in order to ensure a reliable disinfection of all the water carried therein. The catholyte can be discarded by means of line 132.

### COMPARISON EXAMPLE

Production of an electrochemically activated, anodic, dilute water/electrolytic solution (“anolyte”) by means of an apparatus according to FIG. 4 (A) compared with the production of an anolyte using the same apparatus, but accompanied by the bridging of the reverse osmosis plant 106 for lowering the conductivity of the water used and the ion exchanger 104 (B).

### Untreated Water Used:

Electrical conductivity (K_{w}): 543 μS/cm;
total hardness: 14.3° dH (including 10.7° dH carbonate);
pH-value: 7.63.
Untreated Water after Lowering the Electrical Conductivity (A):
Electrical conductivity (K_{w}): 89 μS/cm;
pH-value: 7.25.
Anolyte from (A):
Electrical conductivity: 9820 μS/cm;
free chlorine: 50.6 mg/l (fluctuating);
total chlorine: 56.6 mg/l (fluctuating);
bound chlorine: 6.00 mg/l (fluctuating);
pH-value: 4.00 (fluctuating).
Anolyte from (B):
Electrical conductivity: 3950 μS/cm;
free chlorine: 19.9 mg/l;
total chlorine: 19.9 mg/l;
bound chlorine: <0.05 mg/l;
pH-value: 3.10.

The experiment shows that with the present untreated water with a specific electrical conductivity of approximately 550 μS/cm and a hardness of 14.3° dH (corresponding to 2.560 mmole/l alkaline earth metal ions) without the lowering of the electrical conductivity a pH-value around 3 cannot precisely be set in practice even in the case of relatively high quantities of dosed in sodium chloride solution. The same applies regarding the desired redox potential of approximately 1340 mV vs. SHE. As opposed to procedure (A), the reproducibility in the case of (B) is inferior and the chlorine values are increased to such an extent that there is a danger of them no longer satisfying the German Drinking Water Ordnance. The disinfecting action of the anolyte can consequently not be forecast with absolute reliability.

1-47. (canceled)

48. A method for the production of a disinfectant by electrochemical activation (ECA) of water, wherein an electrolytic solution or a sodium and/or potassium chloride solution is added to the water and the water to which the electrolytic solution has been added in the form of a dilute water/electrolytic solution is supplied with an electrical current in an electrolytic reactor having at least one cathode compartment with a cathode and at least one anode compartment with an anode, the anode being separated spatially from the cathode compartment or separated spatially from the cathode compartment by means of a diaphragm or membrane, wherein a d.c. voltage is applied to the electrodes in order to bring the dilute water/electrolytic solution into a metastable state suitable for disinfection, a pH-value of the dilute water/electrolytic solution in the anode compartment being controlled to a value between 2.7 and 3.5 and a redox potential of the dilute water/electrolytic solution in the anode compartment being controlled to a value between 1240 and 1360 mV, the method comprising the steps of:
a) determining, at a predetermined residence time (Tv) of the water/electrolytic solution in the electrolytic reactor and/or at a predetermined volumetric flow (V′) of the water/electrolytic solution through the electrolytic reactor, a static desired current (Ides, stat) between the electrodes dependent on a composition of the water to be disinfected, the static desired current being determined as a function of a conductivity (κ) and/or hardness (H) of the water according to a straight line equation of form:
Ides,stat=K1*κ+K2
and/or
Ides,stat=K3*H+K4
K1, K2, K3, and K4 being reactor-specific constants; and
b) determining, at a predetermined static desired current (Ides,stat) between the electrodes of electrolytic reactor, a dynamic desired current (Ides, dyn) dependent on a residence time (Tv) of the water/electrolytic solution in the electrolytic reactor and/or a volumetric flow V′ of the water/electrolytic solution through the electrolytic reactor, the dynamic desired current being determined according to a straight line equation of form:
Ides,dyn=K5*Tv+K6
and/or
Ides,dyn=K7*V′+K8
K5, K6, K7 and K8 being reactor-specific constants; and
c) applying a total desired current (Ides, tot) to the electrodes of the electrolytic reactor, which is formed from a sum of the static desired current (Ides, stat) and the dynamic desired current (I des, dyn):
Ides,tot=Ides,stat+Ides,dyn.

49. The method of claim 48, wherein the pH-value of the dilute water/electrolytic solution in the anode compartment is controlled to a value between 2.7 and 3.3 or between 2.8 and 3.2.

50. The method of claim 48, wherein the redox potential of the dilute water/electrolytic solution in the anode compartment is controlled to a value between 1280 and 1360 mV or to a value between 1320 and 1360 mV.

51. The method of claim 48, wherein the pH-value of the dilute water/electrolytic solution in the anode chamber is controlled by controlled addition of a corresponding electrolytic solution quantity.

52. The method of claim 48, wherein the pH-value of the dilute water/electrolytic solution in the anode compartment is controlled by controlling the current flowing between the electrodes.

53. The method of claim 48, wherein the pH-value of the dilute water/electrolytic solution in the anode compartment is controlled by controlling the residence time (Tv) and/or volumetric flow (V′) thereof in or through the electrolytic reactor.

54. The method of claim 48, wherein, for controlling the pH-value of the dilute water/electrolytic solution in the anode compartment of electrolytic reactor to a pH-value between 2.7 and 3.5 at a predetermined desired current between the electrodes of electrolytic reactor, a residence time (Tv) dependent on a composition of the water to be disinfected and/or a volumetric flow (V′) of the dilute water/electrolytic solution in or through electrolytic reactor is determined in dependence on a composition of the water.

55. The method of claim 54, wherein the residence time (Tv) and/or volumetric flow (V′) in or through the electrolytic reactor is determined as a function of a conductivity (κ) and/or hardness (H) of the water.

56. The method of claim 54, wherein the residence time (Tv) and/or volumetric flow (V′) is determined according to a straight line equation of form:
Tv=k1*κ+k2
and/or
V′=k3*H+k4
in which k1, k2, k3 and k4 are reactor-specific constants.

57. The method of claim 54, wherein a quantity of dosed electrolytic solution is kept substantially constant.

58. The method of claim 48, wherein the electrolyte concentration of the dilute water/electrolytic solution added to the electrolytic reactor is controlled to a value of at most 20 g/l.

59. The method of claim 48, wherein a specific electrical conductivity of the water to be electrochemically activated is set to a value of at most 350 μS/cm prior to adding the electrolytic solution.

60. The method of claim 48, wherein control of an electrolyte concentration of an alkali metal chloride concentration of the dilute water/electrolytic solution or a dilute water/alkali metal chloride solution added to the electrolytic reactor takes place by controlling an electrolytic solution quantity added to the water to be electrochemically activated.

61. The method of claim 60, wherein control of the alkali metal chloride concentration of the dilute water/alkali metal chloride solution added to the electrolytic reactor is carried out as a function of a corresponding specific electrical conductivity of the water/alkali metal chloride solution added to the electrolytic reactor, a dependence of the alkali metal chloride concentration on the specific electrical conductivity of the dilute water/alkali metal chloride solution added to the electrolytic reactor being predetermined for the water to be electrochemically activated.

62. The method of claim 61, wherein a dependence of the alkali metal chloride concentration on the specific electrical conductivity of the dilute water/alkali metal chloride solution added to the electrolytic reactor is determined according to a calibration line of the form
κtot=κw+dκ/d[MeCl]*[MeCl]
κtot being the specific electrical conductivity of the dilute water/alkali metal chloride solution added to the electrolytic reactor, κw the specific electrical conductivity of the water to be disinfected, [MeCl] the alkali metal chloride concentration of the dilute water/alkali metal chloride solution and dκ/d[MeCl] the water-specific slope of the calibration line.

63. The method of claim 48, wherein only an electrochemically activated, dilute water/electrolytic solution produced in the anode compartment is used as a disinfectant.

64. The method of claim 48, wherein the method is used for disinfecting water.

65. The method of claim 64, wherein a partial flow is branched off from water to be disinfected, the partial flow is electrochemically activated, and at least the electrochemically activated partial flow in the anode compartment is added as disinfectant to the water to be disinfected.

66. The method of claim 48, wherein the disinfectant is used for disinfecting drinking and service water, rain water, swimming pool water, industrial water, or waste water.

67. The method of claim 48, wherein the disinfectant is used for disinfecting foods, cereals, spices, fruits, vegetables, ice cream, or animal products.

68. The method of claim 48, wherein the disinfectant is used for disinfecting seed.

69. The method of claim 48, wherein disinfectant is used for disinfecting packing containers or packs for hygienic products, foods, pharmaceuticals, or sterile articles.

70. The method of claim 48, wherein the disinfectant is used as an additive for paints, lacquers, varnishes or pigments.

71. The method of claim 48, wherein the disinfectant is used as an additive for coolants or lubricants.

77. The method of claim 48, wherein the disinfectant is used as an additive for fuels, propellants, heating oil, gasoline, or kerosene.
